(19) United States
(12) Patent Application Publication
(10) Pub. No.: US 2004/0235931 A1
(43) Pub. Date: Nov. 25, 2004

(54) Inhibition of herpes virus replication

(76) Inventors: John Docherty, Kent, OH (US); Angelo L. DeLucia, Kent, OH (US)

Correspondence Address: CALFEE HALTER & GRISWOLD, LLP, 800 SUPERIOR AVENUE, SUITE 1400, CLEVELAND, OH, 44114, US

(21) Appl. No.: 10/827,686

(22) Filed: Apr. 19, 2004

Related U.S. Application Data
(60) Provisional application No. 60/463,482, filed on Apr. 17, 2003.

(51) Int. Cl. C12Q 1/70; A61K 31/405

(52) U.S. Cl. 514/415

(57) ABSTRACT

The present invention provides a method of inhibiting the formation of infectious herpes virus particles, particularly infectious herpes simplex virus (HSV) particles, in a host cell. The method involves administering an effective amount of indole-3-carbinol to a herpes virus infected host cell. The present invention also provides a method of treating a herpes virus infection, particularly an HSV infection. The method comprises administering a topical composition comprising a therapeutically effective amount of I3C or a pharmaceutically acceptable salt or ester thereof to a herpes virus-infected site. The present invention also relates to a topical composition for treating a herpes virus infection selected from the group consisting of an HSV infection, a cytomegalovirus infection, and a varicella zoster virus infection.

This application claims the benefit of the filing date of U.S. Provisional Application No. 60/ 463,482, filed Apr. 17, 2003, which is incorporated herein in its entirety.

### BACKGROUND

Human herpes viruses can infect host cells in virtually any organ of the human body. Replication of a herpes virus within an infected host cell leads to lysis of the infected cell and the release of large numbers of infectious virus. The infectious particles released from the lysed cell can infect and destroy other cells at or near the site of the initial infection. These infectious particles can also be transmitted to a non-infected individual. Human herpes viruses can also enter and remain latent, i.e., in the non-replicative state, in other cells of the afflicted individual for life. This life-long infection serves as a reservoir of infectious virus for recurrent infections in the afflicted individual and as a source of infection for an unwitting contact.

At least four of the human herpes viruses, including herpes simplex virus type 1 (HSV-1 ), herpes simplex virus type 2 (HSV-2 ), cytomegalovirus (CMV), and varicella zoster virus (VZV) are known to infect and cause lesions in the eye of certain infected individuals. Together, these four viruses are the leading cause of infectious blindness in the developed world.

HSV-1 primarily infects the oral cavity, while HSV-2 primarily infects genital sites. However, any area of the body, including the eye, skin and brain, can be infected with either type of HSV. Generally, HSV is transmitted to a non-infected individual by direct contact with the infected site of the infected individual.

The initial symptoms of a primary or recurrent HSV infection include tingling, pain, and/or parasthesia at the site of infection. This is followed by formation of a lesion at the infected site, i.e., in the oral cavity, eye, skin, or reproductive tract. Healing typically occurs in approximately ten to fourteen days.

The immune reaction that occurs in response to an HSV infection prevents dissemination of the virus throughout the body of the immmunocompetent individual. Such immune reaction, however, does not eliminate all infectious HSV particles from the body of the afflicted individual. The virus particles that are not killed by the immune response move along the nerve path to the ganglia of the infected individual where they remain in a state of latency. In response to a variety of stimuli including stress, environmental factors, other medications, food additives or food substances, the infectious virus particles may leave the ganglia and cause a recurrent infection at or near the original site of infection. In those HSV-infected individuals who are immunosuppressed or who lack a well-developed immune system, such as neonates, dissemination of the virus particles from the infected site can also occur and lead to life-threatening complications, including encephalitis.

VZV, which is transmitted by the respiratory route, is the cause of chickenpox, a disease which is characterized by a maculopapular rash on the skin of the infected individual. As the clinical infection resolves, the virus enters a state of latency in the ganglia, only to reoccur in some individuals as herpes zoster or “shingles”. The reoccurring skin lesions remain closely associated with the dermatome, causing intense pain and itching in the afflicted individual.

CMV is more ubiquitous and may be transmitted in bodily fluids. The exact site of latency of CMV has not been precisely identified, but is thought to be leukocytes of the infected host. Although CMV does not cause vesicular lesions, it does cause a rash.

There are no known cures for infections with human herpes viruses, i.e., methods of eliminating the virus from the body of the infected individual. In addition, there are very few methods for blocking the formation of infectious herpes virus particles and thereby reducing the frequency, severity, or duration of a herpes virus-induced infection and the likelihood of recurrence of infection in the latently-infected individual. Thus, it is desirable to have additional methods for inhibiting the formation of infectious herpes virus particles. Such methods are useful for limiting the severity of a herpes virus infection within an infected individual and the likelihood of transmission of the herpes virus infection from the infected individual to a non-infected individual.

### SUMMARY OF THE INVENTION

The present invention provides a new method of inhibiting the formation of infectious herpes virus particles, particularly infectious HSV particles, in a host cell. The method involves administering indole 3-carbinol (I3C) or a pharmaceutially acceptable salt or ester thereof, to a host cell that has been infected or will be infected with a herpesvirus. I3C is administered to the host cell in an amount sufficient to inhibit replication of the virus in the virus-infected host cell. Such method is useful for reducing the cytopathic effect of a herpes virus infection. Such method is also useful for preventing the spread of the herpes virus from a virus-infected host cell to a non-infected host cell. Such method is also useful for establishing a model system for studying the molecular events that occur during replication of herpes virus and for studying the factors that trigger replication of a latent herpes virus, particularly replication of latent HSV.

The present invention also provides a method of treating a subject who has a herpes virus infection, particularly an HSV infection, or who has been in contact with or may come in contact with an infectious herpes virus. The method comprises administering a topical composition comprising a therapeutically effective amount of indole 3-carbinol (I3C) or a pharmaceutially acceptable salt or ester thereof, to a herpes virus infected site or to a site that has been in contact with or may come in contact with an infectious herpes virus. The present invention also relates to a topical composition for treating a herpes virus infection selected from the group consisting of an HSV infection, a CMV infection, and a VZV infection.

### BRIEF DESCRIPTION OF THE DRAWINGS

FIG. 1 shows the structures of indole-3-carbinol (I3C) also known as indole-3-methanol.

FIG. 2 is a graph showing the extent of HSV-1 replication in Vero cells treated with varying concentrations of I3C one hour after infection with the virus.

FIG. 3 is a graph showing the extent of HSV-1 replication cells pre-treated with I3C for 6 (3A), 12 (3B), or 24 (3C) hours prior to infection with HSV-1.

FIG. 4 is a graph showing the effect of I3C on replication of acyclovir-resistant HSV-1 in Vero cells treated post infection with the virus or for 24 hours prior to infection with the virus (broken line).

FIG. 5 is a graph showing the effect of 13C on replication of HSV-2 in Vero cells treated post infection with the virus or for 24 hours prior to infection with HSV-2 (broken line).

FIG. 6 is a graph showing the effect of 13C on replication of HSV-1 in MRC-5 cells following infection with the virus or for 12, 24, or 36 hours prior to infection with the virus.

FIG. 7 is a graph showing that incubation of HSV-1 with I3C does not inactivate the virus.

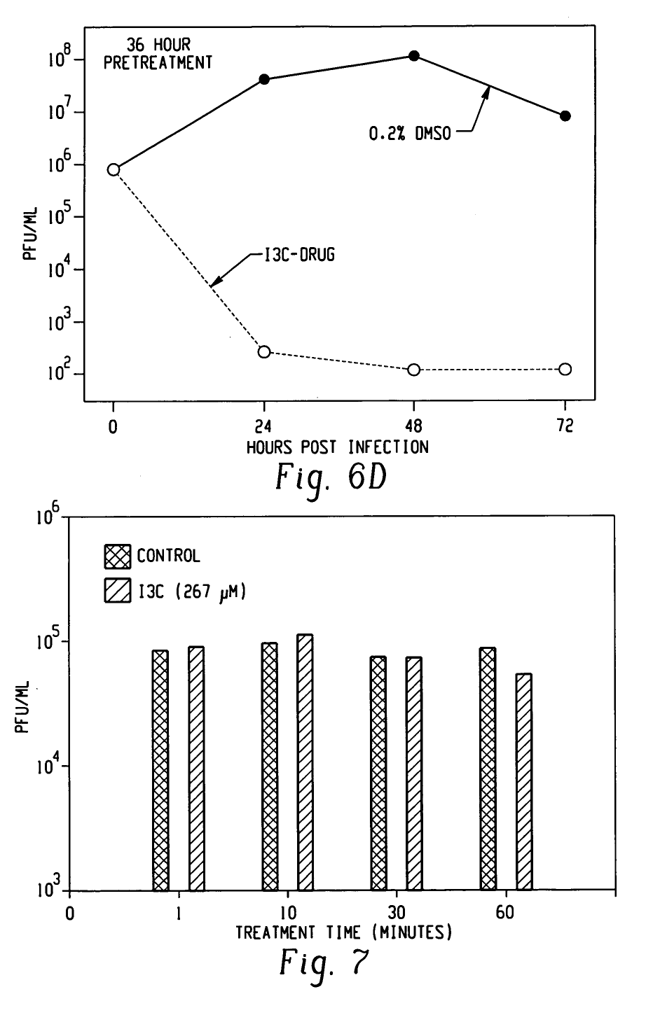

### DETAILED DESCRIPTION OF THE INVENTION

In one aspect, the present invention provides a method of inhibiting formation of infectious herpes virus particles, particularly infectious HSV particles, in a host cell. The method comprises administering indole-3-carbinol (I3C) or a pharmaceutically acceptable salt or ester thereof to the host cell. The I3C is administered in an amount sufficient to or effective to inhibit replication of the herpes virus within the infected cell. Preferably, the I3C , is administered to the host cell either prior to infection of the host cell with the virus or within six hours after infection of the host cell with the virus.

Preferably, the I3C is administered to the host cell by contacting the host cell with or exposing the host cell to a composition comprising the I3C . For example, in vitro, the method comprises adding I3C to the culture medium of herpes virus-infected host cells. In the case of cultured cells, the I3C preferably is added to the medium before the host cells are infected with the virus or within six hours after the host cells are infected with the virus.

It has been determined that treatment of cultured cells in accordance with the present method is non-toxic to cells and blocks replication of HSV at some early stage in the replicative cycle of this human herpes virus. Typical of the herpes viruses, HSV replication occurs in phases, with each phase being dependent on the successful completion of the prior phase. The “immediate early phase” occurs at 1-3 hours after infection and is associated with regulatory and synthetic events. The “early phase” occurs 3-6 hours after infection and is also associated with regulatory and synthetic events, particularly the synthesis of virus DNA. The “late phase” occurs 6-10 hours after infection and is associated with final synthetic events and assembly of viral components into infections virions. Accordingly, since all herpes viruses have in common a replicative scheme that progresses through similar and distinct phases, such method is useful for establishing model systems for studying the molecular events that occur during replication of all herpes viruses. For example, mammalian cell cultures incubated in the presence and absence of I3C may be used to identify cellular factors that are involved in regulating herpes virus synthetic events. Such cell cultures may also be employed to characterize the role of HSV gene products in the replication of infectious virus, particularly those proteins and factors whose function are currently unknown.

Such method is also useful for establishing a model system for studying latency of herpes viruses, particularly latency of the herpes viruses that remain latent in the ganglia, such as for example HSV and VZV. Such model system is useful for characterizing the extracellular factors such as for example hormones and cytokines, as well as the intracellular factors and molecular events that trigger replication of latent herpes viruses.

Methods and Compositions for Treating a Subject with a Herpesvirus Infection

In another aspect, the present invention provides methods for treating a subject with a herpesvirus infection or who may come in contact with infectious herpes virus. The method comprises administering a pharmaceutical composition, preferably a topical composition, comprising a therapeutically effective amount of I3C or a pharmaceutically acceptable salt or ester thereof to the site of the infection. As used herein “site of the infection” means a previously uninfected site which may come into contact with infectious herpes virus, has come in contact with infectious herpes virus , or the site of a current or prior herpes virus-induced lesion. Such method is particularly useful for treating local herpes virus infections, such as for example, HSV-induced skin lesions, HSV-induced eye infections, HSV-induced lesions of the reproductive tract, CMV-induced eye lesions, and VZV-induced eye or skin lesions. In such cases, it is preferred that the I3C be applied directly to the infected site. It is preferred that the I3C be administered to the herpesvirus-infected site in the form of an aqueous solution or in the form of a salve. For eye infections, it is preferred that an aqueous solution of the I3C be administered as an eye drop. For herpesvirus skin lesions, such as for example, HSV-induced skin lesions, or HSV-induced lesions of the reproductive tract, it is preferred that the composition be applied topically.

### Indole-3-Carbinol

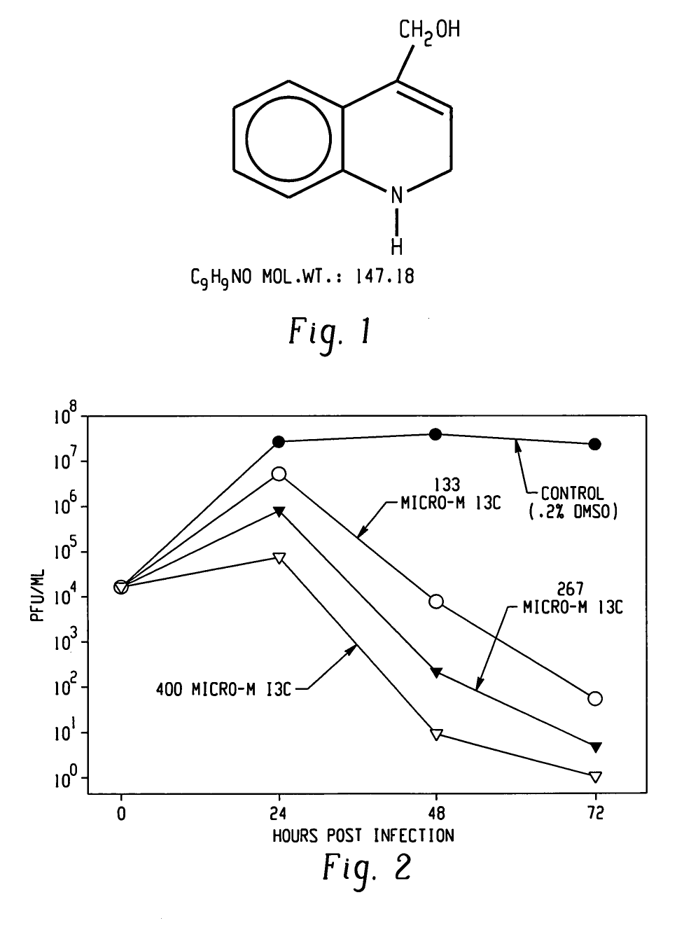

The structural skeleton of the compound employed in the present invention, i.e., I3C is an indole to which is attached a methanol group (See FIG. 1). The methanol group is attached to carbon 3 of the I3C indole. I3C is a naturally occurring component of Brassica vegetables, such as cabbage, broccoli, and brussels sprouts. I3C is commercially available.

### Topical Composition

The pharmaceutical composition comprises a therapeutically effective amount of I3C or a pharmaceutically acceptable salt or ester thereof, and a pharmaceutically acceptable carrier, preferably a topical carrier. Preferably, the composition comprises a relatively inert topical carrier. Many such carriers are routinely used and can be identified by reference to pharmaceutical texts. Examples include polyethylene glycols, polypropylene copolymers, and some water soluble gels. Such a composition, referred to hereinafter as the “topical composition”, may also contain diluents, fillers, salts, buffers, stabilizers, solubilizers, and other pharmaceutically acceptable materials well known in the art. The term “pharmaceutically acceptable” means a non-toxic material that does not interfere with the effectiveness of the antiviral or antibacterial activity of the I3C.

In practicing the present method of treatment or use, a pharmaceutical composition comprising a therapeutically effective amount of I3C is applied to the site of infection in the host subject before or after the host subject is exposed to the virus or bacterium. Such composition is particularly effective in treating infections of the eye, oral cavity and vagina as well as border areas of the lips and rectum. In the case of oral administration, dentrifices, mouthwashes, tooth paste or gels, or mouth sprays are used. Vaginal or rectal administration may be by the usual carriers such as douches, foams, creams, ointments, jellies, and suppositories, the longer lasting forms being preferred. Ocular administration is preferably by ophthalmic ointments or solutions. Lip treatment is, preferably, in the form of a gel.

The topical composition may further contain other agents which either enhance the activity of the I3C or complement its activity or use in treating the viral disease or bacterial disease. Such additional factors and/or agents may be included in the pharmaceutical composition to produce a synergistic effect with the I3C, or to minimize side effects. The topical composition may also contain an agent which enhances uptake of the I3C through the skin.

Preferably the topical composition comprises a solvent for I3C, such as, for example, an alcohol. A liquid carrier such as water, petroleum, oils of animal or plant origin such as peanut oil, mineral oil, soybean oil, or sesame oil, corn oil, or synthetic oils may be added. The liquid form of the pharmaceutical composition may further contain a physiological saline solution, dextrose or other saccharide solution, or glycols such as ethylene glycol, propylene glycol or polyethylene glycol. The preparation of such topical composition having suitable pH, isotonicity, and stability, is within the skill in the art.

The topical composition of the invention may be in the form of a liposome in which I3C or the pharmaceutically acceptable salt or ester thereof is combined with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art.

### Dosage

The I3C or the pharmaceutically acceptable salt or ester thereof is administered to the site of infection in the host subject in a therapeutically effective amount. As used herein, the term “therapeutically effective amount” means the total amount of I3C that is sufficient to show a meaningful benefit, i.e., treatment, healing, prevention, amelioration, or reduction in the symptoms of the herpesvirus infection or an increase in rate of healing, amelioration or reduction in the symptoms of such infection.

By “treating” is meant curing or ameliorating a herpevirus infection or tempering the severity of the infection. By preventing is meant blocking the formation of a primary lesion or recurrence of a lesion at the infected site. The dosages of the I3C , which can treat or prevent an HSV, VZV, CMV infection can be determined in view of this disclosure by one of ordinary skill in the art by running routine trials with appropriate controls. Comparison of the appropriate treatment groups to the controls will indicate whether a particular dosage is effective in preventing or treating the infection at the levels used in a controlled challenge.

It is contemplated that the various pharmaceutical compositions used to practice the method of the present invention should contain about 0.1 μg to about 100 mg/ml of I3C. Although a single application of the topical composition may be sufficient to ameliorate the pathological effects of the herpesvirus, it is expected that multiple doses will be preferred.

### Delivery

Administration of the pharmaceutical composition is via local administration to the infected site. In those individuals who have experienced a primary lesion, it is preferred that the topical composition be applied at the prodromal stage of infection, i.e., during early symptoms of pain, tingling, parasthesia. Preferably, the composition is applied to the site of infection periodically, more preferably every three hours. The duration of therapy using the pharmaceutical composition of the present invention will vary, depending on the severity of the disease being treated and the condition and response of each individual patient. Ultimately the attending physician will decide on the appropriate duration of using the pharmaceutical composition of the present invention.

The following examples are for purposes of illustration only and are not intended to limit the scope of the claims which are appended hereto. All references cited herein are specifically incorporated in their entirety herein.

### EXAMPLES

Materials and Methods

1. Cell Lines:

African green monkey kidney cells (Vero) and Human neonatal lung fibroblasts cells (MRC-5) were obtained from the American Type Culture Collection, (Rockville, Md.). Vero cells were grown and maintained in Medium 199 supplemented with 5% fetal bovine serum, 0.075% NaHCO₃ and 50 μg/ml gentamycin sulfates. MRC-5 cells were grown and maintained in Minimal Essential Medium with Earle's Balanced Salt Solution containing 2 mM L-glutamine supplemented with 10% fetal bovine serum, 0.lmM nonessential amino acids, 1 mM sodium pyruvate, 2% sodium bicarbonate and 50 μg/ml gentamycin sulfate.

2. Cell-Doubling Studies

Trypan blue exclusion and cell clone counting methods were used to determine cell-doubling times. Untreated Vero and MRC-5 cells were counted at 24, 48 and 72 hour time points for the trypan blue exclusion and clone counting methods. Two samples for each cell type and time point was used. The equation for geometric or exponential growth was used to quantify cell division and values calculated by a Statistical Analysis Software program. Vero cell doubling time was determined to be ˜24 hours and MRC-5 cell doubling time was determined to be ˜48 hours.

3. Chemicals

I3C was purchased from Sigma Chemical Company (St. Louis, Mo.). Stock concentrations of I3C were prepared in 0.2% dimethyl sulfoxide (DMSO) and diluted to final concentrations in tissue culture media. Viral replication studies in media containing 0.2% DMSO established that there was no effect on virus replication when compared to virus replication in media lacking 0.2% DMSO.

4. Toxicity Studies

Cells were exposed to drug treated media of varying concentrations. Drug cytotoxic concentrations were determined by MTT toxicity assays (J. Tissue Culture Methods. 11:1, 15, 1988) and resulted in a CD₅₀ of ˜500 μM taken at 24, 48 and 72 hours. I3C concentrations used in these studies were 50% and 75% of the CD₅₀. Cells were seeded onto a 96 well at 10,000 cells/well and grown for 24 hours. Twelve different serially diluted concentrations of I3C were then incubated with the cells for 24, 48 and 72 hours and colorimetric readings were taken at these time points using the MTT reagent. Controls included in the 96 well plate were, media alone that contained no cells but with I3C, media with cells but no I3C, and plain media with 0.2% DMSO and no cells or drug. At 24, 48 and 72 hours a sigmoidal curve was generated for each time period and the data analyzed. Approximately, 500 μM was the concentration at which 50 percent of the cells died (CD₅₀). This was observed in Vero and MRC-5 cells. The CD₅₀ for both cell types was analyzed at 48 hours because maximum replication of herpes simplex generally occurs at this time.

### Example 1

### Inhibiting Formation of Infectious HSV-1 Particles in Vero Cells by Post-Infection Treatment with I3C

Cultures of African green monkey kidney cells (Vero) cells, obtained from the American Type Culture Collection, Rockville, Md., were grown to confluence in Medium 199 supplemented with 5% fetal bovine serum, 0.075% NaHCO₃, and 50 μg/ml gentamycin sulfate in 25 cm² tissue culture flasks. Cells were infected with HSV-1 at a multiplicity of infection (moi) of one and incubated at room temperature for one hour to allow for virus attachment to and penetration of the cell. Under these conditions, approximately half of the cells are infected with virus. Thereafter, the cultures were rinsed three time with media and incubated in medium containing 133 μM, 267 μM, or 400 μM I3C prepared in 0.2% dimethyl-sulfoxide (DMSO). Controls were treated identically, but were incubated without the I3C.

Upon addition of the medium to the cultures and at 24 hours time periods thereafter, i.e., 0 hours, 24 hours, 48 hours, and 72 hours after addition of the drug, cells and medium were frozen at −70° C. Samples were then thawed, sonicated and titrated on Vero cells to determine the number of plaque forming units (pfu's) of virus produced by each culture.

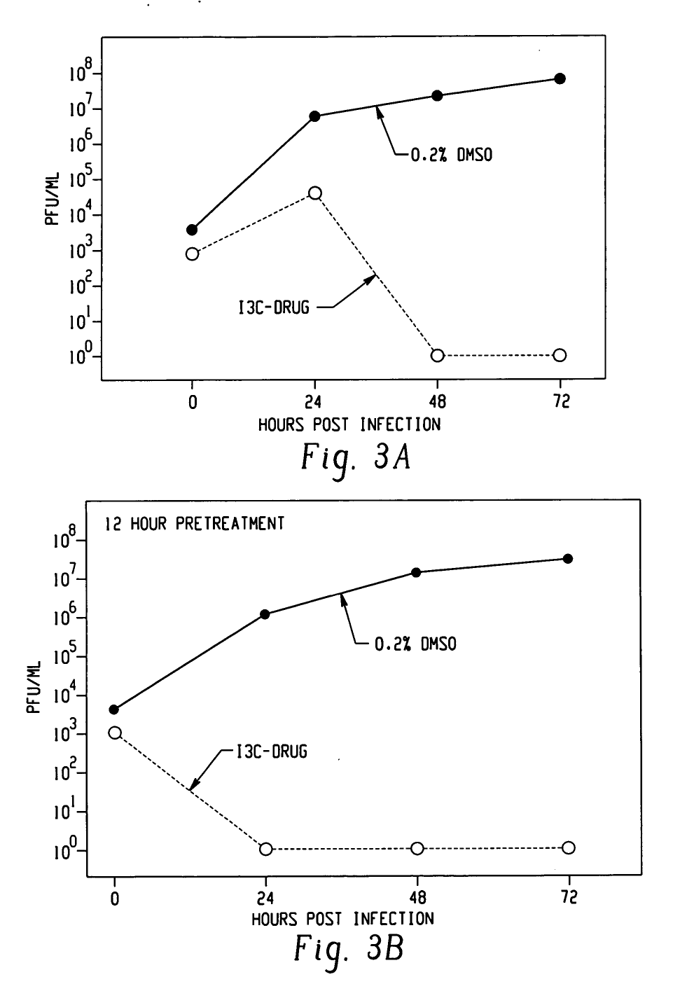

As shown in FIG. 2, the number of pfu's produced in the control cultures infected with an moi of 1 reaches peak production at approximately 24 hours after infection. At this time, the system is exhausted, i.e., active virus has infected and destroyed not only those cells infected during the initial one hour of incubation but also those cells which became infected with virus released by the initially-infected cells. The lack of increase observed in the control cultures at 72 hours treatment indicates that the virus production has peaked, due to the lack of viable cells in which to reproduce.

As shown in FIG. 2, treatment of cells with 400 μM I3C inhibited formation of infectious virus particles in HSV-1 infected cells by more than 99% at 24 hours. By 72 hours, infectious HSV particles were virtually undetectable in cultures continuously incubated in the presence of 400 μM I3C . These results also demonstrate that inhibition of virus replication by I3C is dose dependent.

### Example 2

### Inhibiting Formation of Infectious HSV-1 Particles by Contacting Vero Cells with I3C Prior to Infection

Vero cell cultures were infected with HSV-1 as described above in Example 1 except that the cells were treated with 267 μM of I3C for 6, 12, or 24 hours prior to infection. Cells were infected with HSV-1 at an moi of 1 for 1 hour. Following removal of the unabsorbed virus, the infected cells were incubated in media containing 0.2% DMSO (control cells) or in media containing 267 μM of I3C (test cells) At 24, 48, and 72 hours after infection, the number of pfu's present in the cells and medium of the I3C -treated cultures was determined.

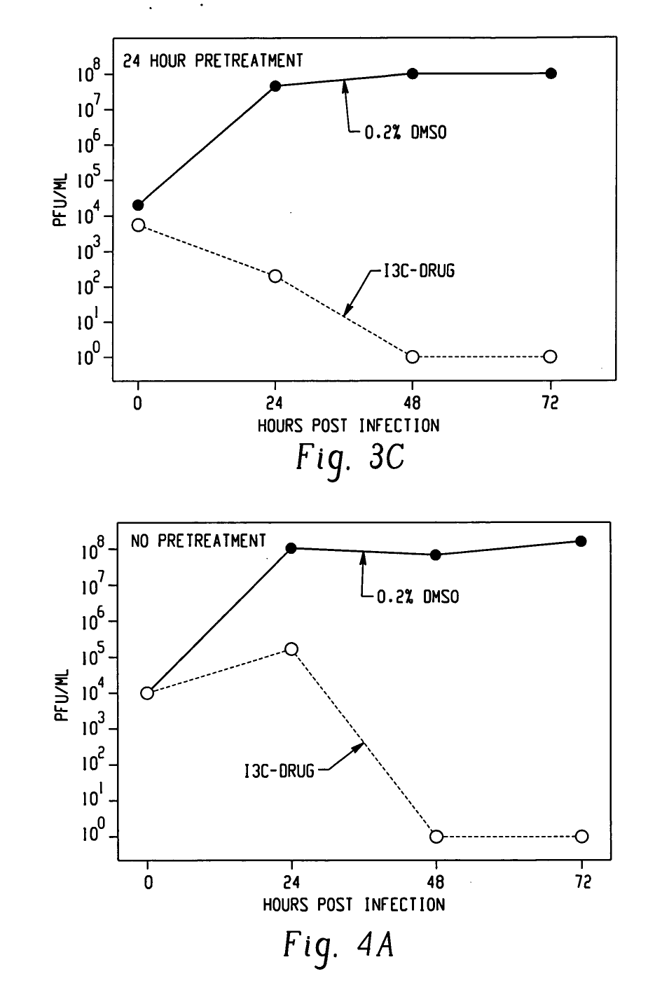

As shown in FIG. 3, inhibition of HSV replication was dramatic and complete when cells were incubated in I3C containing medium for 12 to 24 hours prior to infection. When cells were pre-incubated in I3C-containing medium for 6 hours prior to infection, HSV replication proceeded at a reduced rate for 24 hours and then rapidly decreased at 48 and 72 hours to undetectable levels.

### Example 3

### Inhibiting Formation of Acyclovir-Resistant Infectious HSV-1 Particles by Contacting Vero Cells with I3C Prior to and Post Infection

Vero cell cultures were infected with acyclovir resistant HSV-1 at an moi of one for one hour. Test cells were treated with 267 μM of I3C immediately after or 24 hours prior to infection. At 24, 48, and 72 hours after infection, the number of pfu's present in the test cell pre-treated with I3C or treated with I3C post infection were determined.

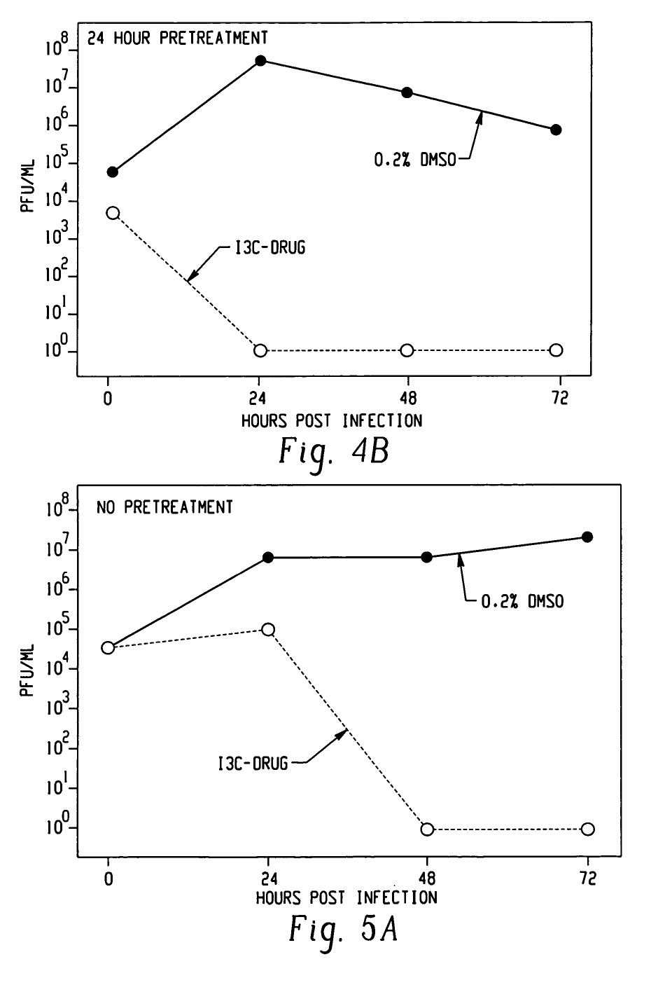

As shown in FIG. 4, inhibition of acyclovir-resistant HSV 1 replication was dramatic and complete when cells were incubated in I3C containing medium for 24 hours prior to infection. When cells were incubated in I3C-containing medium post infection with acyclovir-resistant HSV 1, replication of the acyclovir-resistant HSV proceeded at a reduced rate for 24 hours and then rapidly decreased at 48 and 72 hours to undetectable levels.

### Example 4

### Inhibiting Formation of Infectious HSV-2 Particles by Contacting Vero Cells with I3C Prior to and Post Infection

Vero cell cultures that had been pretreated with 267 μM of I3C were infected with HSV-2 at an moi of one for 1 hour At 24, 48, and 72 hours after infection, the number of pfu's present in the cells and medium of the I3C pre-treated cultures were determined. In addition, Vero cells were infected with HSV-2 at an moi of one for 1 hour, and thereafter the cultures were rinsed three time with media and incubated in medium containing 267 μM prepared in 0.2% dimethyl-sulfoxide (DMSO). At 24, 48, and 72 hours after infection, the number of pfu's present in the test cell pre-treated with I3C or treated with I3C post infection were determined.

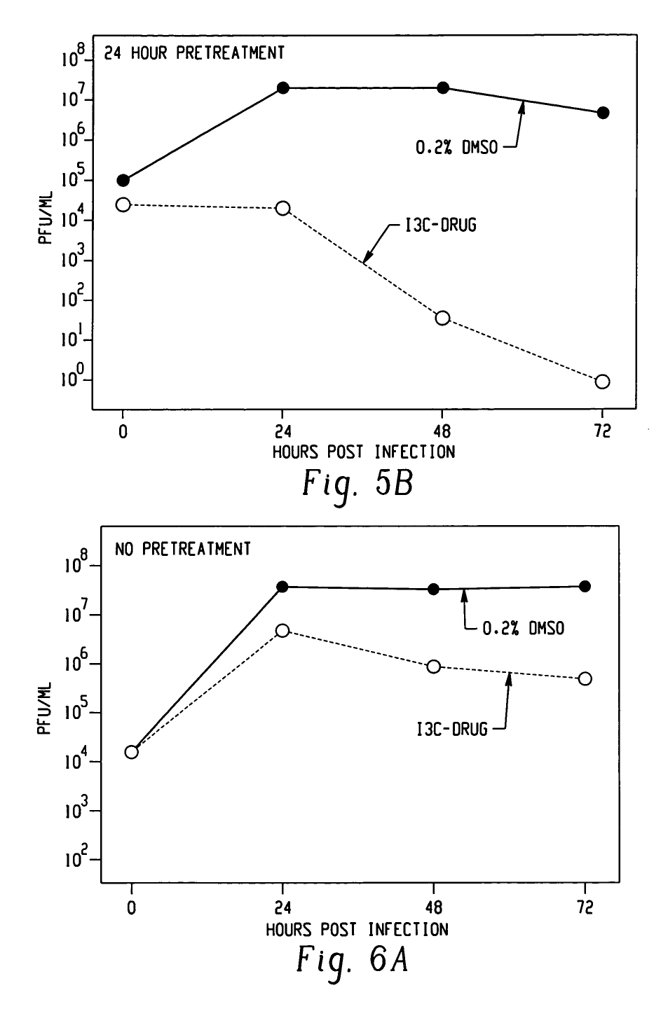

As shown in FIG. 5, inhibition of HSV-2 replication proceeded at a reduced rate for 24 hours in cells that had been treated with I3C for 24 hours pre-infection and for 24 hours post infection. Thereafter, HSV-2 replication decreased at 48 and 72 hours in both the pre-treated and post-treated Vero cells.

### Example 5

### Inhibiting Formation of Infectious HSV-1 Particles in MRC-5 Cells by Contacting with I3C Prior to and Post Infection

MRC-5 cell cultures were infected with HSV-1 as described above in Example 1 except that the cells were treated with 375 μM of I3C following infection or for 12, 24, or 36 hours prior to infection. Cells were infected with HSV-1 at an moi of 1 for 1 hour. Following removal of the unabsorbed virus, the infected cells were incubated in media containing 0.2% DMSO (control cells) or in media containing 375 μM of I3C (test cells) At 24, 48, and 72 hours after infection, the number of pfu's present in the cells and medium of the cultures treated with I3C prior to or after infection was determined.

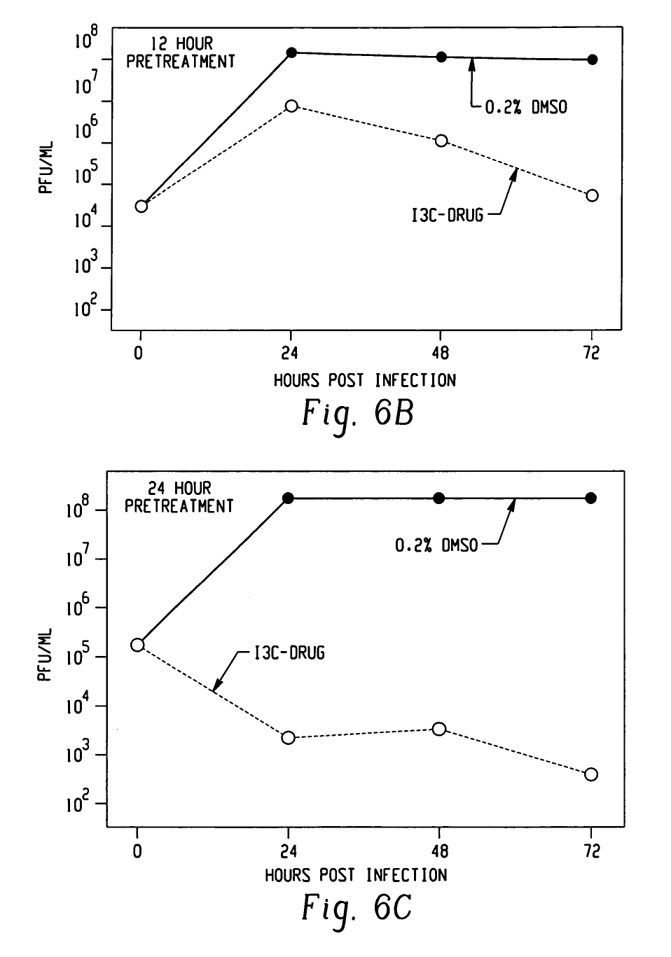

As shown in FIG. 6, pretreatment of MRC5 cells with 375 μM of I3C for 24 or 36 hours prior to infection with HSV-1 results in inhibition of HSV-1 replication in these cells. It is believed that this delay in I3C 's inhibitory effect on HSV-1 replication is due to the longer cell cycling time of the MRC5 cells.

### Example 6

### I3C Does Not Directly Inactivate HSV

To determine if I3C could directly inactivate I3C studies were done in which HSV was mixed with a solution of I3C (267 μM). Samples were taken at 1, 10, 30 and 60 minutes and the presence of infectious virus determined by the plaque assay. The results presented in FIG. 4 demonstrate that when compared to control virusl which was not incubate with I3C, there was no significant inactivation of HSV. The results suggest that I3C does not reduce infection HSV by direct inactivation.

What is claimed is:

1. A method of inhibiting formation of infectious herpes virus particles in a host cell comprising:
administering indole-3-carbinol (I3C) or a salt or ester thereof to the host cell.

2. The method of claim 1 wherein I3C or the salt or ester thereof is administered prior to or within 6 hours of infection of the host cell with the herpes virus.

3. The method of claim 1 wherein I3C or the salt or ester thereof is administered to the cells for a period of from 12 to 36 hours prior to infection of the host cell with the herpes virus.

4. The method of claim 1, wherein the herpes virus is HSV-1.

5. The method of claim 1, wherein the herpes virus is acyclovir-resistant HSV-1.

6. The method of claim 1, wherein the herpes virus is HSV-2.

7. A method of treating a subject having an infection induced by one or more herpes virus selected from the group consisting of HSV-1, acylovir-resistant HSV-1, HSV-2, CMV and VZV or who may come in contact with said one or more herpes viruses, said method comprising administering a pharmaceutical composition comprising a therapeutically effective amount I3C or a pharmaceutically acceptable salt or ester thereof to the subject, wherein the pharmaceutical composition is applied at or proximate a known site of infection or a site which has the potential to come in contact with said one or more infectious herpes virus.

8. The method of claim 7 wherein the pharmaceutical composition further comprises a topical carrier and administration is by topical administration to the skin.

9. The method of claim 7 wherein administration is to the eye.

10. The method of claim 7 wherein administration is to the oral cavity or lips.

11. The method of claim 7 wherein administration is by vaginal insertion or anal insertion.

12. The method of claim 7 wherein the herpes virus infection is caused by HSV-1, acyclovir-resistant HSV-1, or HSV-2.

13. The method of claim 7 wherein the pharmaceutical composition is administered to an infected site during the prodromal stage of infection.

14. A topical composition for reducing the symptoms of a herpes virus infection, said herpes virus being selected from the group consisting HSV-1, acyclovir-resistant HSV-1, HSV-2, VZV and CMV, said topical composition comprising:
(a) a therapeutically effective amount of I3C or a pharmaceutically acceptable salt or ester thereof; and
(b) a topical carrier.

15. A unit dosage form of I3C adaptable for topical administration, comprising an amount of I3C effective to relieve a symptom of HSV-1, acyclovir-resistant HSV-1, HSV-2, or a combination of said herpes viruses.

16. The unit dosage form of claim 15, wherein said form fuirther comprises labeling indicating that multiple doses of said unit dosage form are to be applied periodically to the site of infection.
